(19) United States
(12) Patent Application Publication
(10) Pub. No.: US 2005/0208678 A1
(43) Pub. Date: Sep. 22, 2005

(54) Anti-fusion assay

(76) Inventors: Dong Xie, Germantown, MD (US); John W Erickson, Frederick, MD (US); Paul Grulich, Gaithersburg, MD (US)

Correspondence Address: Dianne B Elderkin;Woodcock Washburn, One Liberty Place - 46th Floor, Philadelphia, PA, 19103, US

(21) Appl. No.: 10/490,716

(22) PCT Filed: Sep. 27, 2002
(86) PCT No.: PCT/US02/30611

§ 371 Date: Mar. 25, 2004

Related U.S. Application Data
(60) Provisional application No. 60/324,948, filed on Sep. 27, 2001.

(51) Int. Cl. G01N 33/561; G06F 19/00; G01N 33/48; G01N 33/50

(52) U.S. Cl. 436/516; 702/19

(57) ABSTRACT

Methods of identifying a fusion inhibitor and inhibitors of gp41-mediated membrane fusion are disclosed. The methods comprise, for example, providing a first helical polypeptide comprising a sequence of IQN17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; measuring, by capillary zone electrophoresis, the degree of complex formation between these peptides; and comparing the measured degree of complex formation to the degree in the presence of a test composition.

### BACKGROUND OF THE INVENTION

Current therapy for the treatment of human immunodeficiency virus (HIV) generally targets the reverse transcriptase and protease. However, several other gene products of the HIV virus, such as the envelope glycoprotein, also play critical roles in infection.

This glycoprotein consists of two non-covalently associated subunits, gp120 and gp41, generated by proteolytic cleavage of the precursor gp160 protein. It resides in the viral membrane as a complex of three gp120 and three gp41 subunits. It is the gp41 subunit that mediates fusion of the membranes of the virus and target cell, allowing the HIV virus to infect new cells. The gp120 subunit is involved in target cell recognition and receptor binding.

The process of membrane fusion mediated by gp41 involves a conformational change in the glycoprotein, exposing in the target cell membrane a trimeric coiled coil formed by alpha helices from the N-terminal region of each of the three gp41 subunits (the N-helix). This coiled coil interacts with alpha helices from the C-terminal region of the three-gp41 subunits (the C-helix), imbedded in the viral membrane. The resulting hexameric alpha helical interaction between the N-helix and the C-helix regions of gp41 fuses the viral and cellular membranes.

Proteolytic studies were used to identify the gp41 segments responsible for formation of the hexameric fusion intermediate, called N36 and C34. (D. C. Chan et al., Cell 89:263-273 (1997); incorporated herein by reference). Intriguingly, residues within the N36 and C34 regions are some of the most highly conserved residues of the envelope coding region of the HIV genome. Several mutations that inhibit membrane fusion and abolish infectivity map to these regions. Moreover, nanomolar concentrations of synthetic peptides corresponding to these regions have been shown to inhibit HIV infectivity and syncytia formation in cell culture, suggesting that they act as inhibitors of gp4′-mediated membrane fusion. These results indicated that an isolated complex between peptides comprising amino acids sequences from the N36 and C34 regions would be an excellent model of the gp41 fusogenic intermediate.

Synthetic N36 and C34 peptides were shown to form a stable hexameric structure under appropriate conditions in vitro, indicating that peptides containing amino acid sequences from these regions can form the hexameric gp41 core in the absence of the remainder of the gp41 subunit. The X-ray crystal structure of the N36/C34 complex was determined by D. C. Chan et al., Cell 89:263-273 (1997). The structure revealed the three C34 peptides representing the C-helix of gp41 were packing in an antiparallel fashion against the three N36 peptides representing the N-helix, forming a six-helix bundle. The structure is similar to those of membrane fusion intermediates from the influenza and Mo-MLV viruses, a further indication that the C34/N36 complex is a good model for a membrane fusion intermediate.

The interactions between C34 and N36 mainly involve residues at the a and d positions of C34 and the e and g positions of N36. In particular, the N-helices form an interior, trimeric coiled coil with three hydrophobic grooves. The hydrophobic cavities are filled by three residues of the C-helix, Trp 628, Trp 631, and Ile 635. In contrast, residues of the C-helix such as Met 629, Gin 630, and Arg 633, located at the b, c, and f positions, lie on the outside of the hexamer, and make no contacts with the N-helix.

The N-helix residues that form the hydrophobic pocket (residues 565-577) are highly conserved among HIV strains. The RNA that encodes these residues is also part of the Rev-response element, a highly structured RNA critical for the viral lifecycle.

The C34 peptide has been shown to be a potent inhibitor of HIV infectivity and membrane fusion. Alanine mutagenesis studies on C34 showed that mutation of the residues corresponding to Trp 628, Trp 631, or Ile 635 to alanine had significant effects on both C34 inhibition of membrane fusion and on complex formation with N36. Alanine mutation of either Met 629 or Arg 633, which do not contact the N-helix, had no significant effect on either membrane fusion or on C34/N36 complex formation. (D. C. Chan et al., Proc. Natl. Acad. Sci., U.S.A. 95:15613-7 (1998); incorporated herein by reference.) These data indicated that the hydrophobic interactions involving residues 565-577 of the N-helix and residues Trp 628, Trp 631, and Ile 635 of the C-helix play an important role in stabilizing the hexameric alpha helical bundle for membrane fusion.

This hydrophobic interaction is an attractive target for candidate gp41 inhibitors. While this interaction is present in the N36/C34 peptide complex, this complex is not ideally suited for screening of potential gp41 inhibitors because N36 is insoluble and subject to aggregation in the absence of C34. (M. Lu et al. Nat. Struct Biol. 2: 1075-1082; D. M. Eckert et al. Cell 99: 104). Therefore, a soluble fusion peptide comprising the C-terminal 17 residues of N36 and 29 residues of GCN4-pI_{Q}I was constructed (with a 1 residue overlap between the two regions, making the peptide 45 residues long). (D. M. Eckert et al. Cell 99: 105; incorporated herein by reference). This peptide, called IQN17, has the sequence RMKQIEDKIEEIESKQKKIENEIARIKKLLQLTVWGIKQLQARIL (SEQ ID NO: 1), with the gp41 sequence of amino acids 565-581 underlined. IQN17 includes 3 mutations of surface residues in the GCN4-pI_{Q}I region to improve solubility. It is fully helical, with nearly the same superhelix parameters as the gp41 N-helix, and forms a stable trimer in solution. The X-ray crystal structure of IQN17 in complex with a cyclic peptide (D-peptide) showed that the hydrophobic pocket formed by residues 565-577 is nearly identical to that of N36. (WO 00/06599).

International application WO 00/06599 describes the use of IQN17 as a mimic of the N-helix region of gp41 for identification of candidate D-peptide membrane fusion inhibitors. A library of D-peptide molecules designed to present hydrophobic moieties into the binding pocket of IQN17 were tested in screening assays using mirror image phage display. However, to date, the use of IQN17 has been limited to D-peptides. Further, WO 00/06599 does not provide a simple assay for identifying anti-fusion compounds.

On another aspect, goals of drug design initially comprise the characterization of selectivity and affinity, efficacy, toxicity (therapeutic window/safety margin), pharmacokinetics, and stability, for a given compound. One of the earlier steps in drug design and discovery is focused on discriminating potential active compounds amongst a large variety of chemical compounds. This discrimination of potential active chemical structures is suitably accomplished by ligand-binding assays. Ligand-binding assays measure the affinity and/or degree of a drug to remain associated with a receptor. Within a molecule structure, the affinity or degree of binding of a specific moiety for a target recognition site of the receptor is particularly useful information in designing and optimizing effective compounds against a given target. Additionally, ligand-binding assays are especially convenient in elucidating mechanisms of action or inhibition.

Jiang et al. (J Med Chem 1999 Aug. 26;42(17):3203-9) describe an ELISA assay utilizing antibody that recognizes the complex of N36 and C34. In the assay, N36 is pre-incubated with screening compounds prior to the addition of C34 peptide. The extent of N36/C34 complex formation is accessed by antibody for identification of inhibitors of complex formation process.

Whereas much is known about the relationships between sequence and activity for certain peptide inhibitors (see Wild et al, 1992, PNAS 89; Wild et al, 1994, PNAS 91; Jiang et al., 1993, Nature, 365; Wild et al., 1995, AIDS, Res. Hum. Retroviruses, 11; Neurath et al., 1995, Res. Hum. Retroviruses, 11), the mechanism of inhibition has not yet been fully established for many fusion inhibitors.

In a study by Ryu et al., Biochemical and Biophysical Research Communications, 1999, 265, a C51 peptide was bound tightly to Trx-N, increasing the solubility of Ec-gp41ec. The naDP178 showed very weak binding affinity to Trx-N, however, it effectively solubilized Ec-gp41 ec. C27 peptide showed significant binding to Trx-N; however, it did not affect the solubility of Ec-gp41 ec.

In another example by Cole et al., Biochemistry, 2001, 40, results from in vitro affinity of certain D-peptides (D10-p1-2K, D10-p5-2K, D10-p4-2K) for 10N17, measured by isothermal titration calorimetry, did not correlate to results in vivo.

Although screening assays for fusion inhibitors have been available for some time, mechanistic studies of HIV fusion inhibitors targeted to gp41 have been hampered by the lack of sensitive methodologies, which may discriminate between a large range of degrees of binding, leaving a tremendous range of chemical diversity unsampled.

A model and the means for directly measuring ligand-binding with high sensitivity and robustness is desired for screening a broader window of anti-fusion compounds. Additionally, a method that does not rely on anti-bodies or that may be effective without the use of antibodies may be desired for some applications.

### SUMMARY OF THE INVENTION

Surprisingly, it has been found that C-terminal peptides, in particular C28 and C34, can bind to IQN17, forming a complex that mimics the in vivo pre-hairpin intermediate structure of gp41. As such, it has been discovered that IQN17 in combination with a C-helix peptide containing Trp 628, Trp 631, and Ile 635 is a useful model for identifying candidate gp41-mediated membrane fusion inhibitors of a variety of chemical compositions. Such a complex specifically presents the important hydrophobic interactions of the gp41 hexameric membrane fusion intermediate in correct geometry such that the interactions can be targeted by a variety of competitor molecules. Such a complex is not obvious from prior art.

Additionally to the use of IQN17 in combination with a C-helix peptide containing Trp 628, Trp 631, and Ile 635, the employment of a capillary zone electrophoresis (CZE) means of measuring, has advantageously increased the sensitivity of this screening assay, allowing the detection and discrimination of a broader spectra of compounds, leads, key moieties which could be useful for designing new potent HIV fusion inhibitors. In addition, capillary zone electrophoresis is a rapid, facile, versatile detection technique, which requires small amounts of sample volumes. Although there are other techniques known in the art that measure binding, such as fluorescence polarization, fluorescence resonance energy transfer etc., we found CZE is particularly suitable for screening inhibitors of the IQN17/C-helix complex formation. This is due to the relatively weak binding between IQN17 and C-helix peptides of gp41, which results in a low signal to noise ratio for the traditional methods. In CZE, however, the IQN17 bound form and the free form of C-helix peptide is separated while their relative concentration is fixed because of the high electric static pressure inside of the capillary. This allows an almost zero background and an accurate determination of the degree of IQN17/C-helix complex formation in the presence of screening compounds. Optionally the use of capillary zone electrophoresis combined with laser-induced fluorescence detection further increases said sensitivity.

As a result, one embodiment of this invention involves a method of identifying a fusion inhibitor comprising: providing a first helical polypeptide comprising a sequence of IQN17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline (proline disrupts alpha-helix formation); providing a test composition; measuring, by capillary zone electrophoresis, the degree of complex formation between the first helical polypeptide and the second helical polypeptide in the presence of the test composition; and comparing the measured degree of complex formation to that between the first helical polypeptide and the second helical polypeptide in the absence of the test composition to determine if the test composition is a fusion inhibitor.

Another embodiment of this invention involves a method of identifying a fusion inhibitor comprising: providing a first helical polypeptide consisting essentially of the sequence of IQN17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; providing a test composition; measuring, by capillary zone electrophoresis, the degree of complex formation between the first helical polypeptide and the second helical polypeptide in the presence of the test composition; and comparing the measured degree of complex formation to that between the first helical polypeptide and the second helical polypeptide in the absence of the test composition to determine if the test composition is a fusion inhibitor.

Also within the scope of the invention is a method of identifying inhibitors of gp41-mediated membrane fusion, comprising: providing a first helical polypeptide comprising a sequence of IQN17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; providing a test composition; measuring, by capillary zone electrophoresis, the degree of complex formation between the first helical polypeptide and the second-helical polypeptide in the presence of the test composition; and comparing the measured degree of complex formation to that between the first helical polypeptide and the second helical polypeptide in the absence of the test composition; wherein a reduction in the degree of complex formation between the first helical polypeptide and the second helical polypeptide in the presence of the test composition compared to the degree of complex formation between the first helical polypeptide and the second helical polypeptide in absence of the test composition identifies the test composition as an inhibitor of gp41-mediated membrane fusion.

The invention also includes a method of identifying inhibitors of gp41-mediated membrane fusion, comprising: providing a first helical polypeptide consisting essentially of the sequence of 10N17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; providing a test composition; measuring, by capillary zone electrophoresis, the degree of complex formation between the first helical polypeptide and the second helical polypeptide in the presence of the test composition; and comparing the measured degree of complex formation to that between the first helical polypeptide and the second helical polypeptide in the absence of the test composition; wherein a reduction in the degree of complex formation between the first helical polypeptide and the second helical polypeptide in the presence of the test composition compared to that between the first helical polypeptide and the second helical polypeptide in absence of the test composition identifies the test composition as an inhibitor of gp41-mediated membrane fusion.

The present invention further comprises a method of identifying the mechanism of inhibition of a fusion inhibitor comprising: providing a first helical polypeptide consisting essentially of the sequence of IQN17 (SEQ ID NO: 1); providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; providing a test composition; measuring, by capillary zone electrophoresis, the degree of complex formation between the first helical polypeptide and the second helical polypeptide in the presence of the test composition; and comparing the measured degree of complex formation to that between the first helical polypeptide and the second helical polypeptide in the presence of a different test composition, to determine if the first test composition has the same or different mechanism of inhibition.

In a particular embodiment the present invention may also be applied as a method for measuring resistance of mutant gp41 fusion proteins to a test composition comprising: providing a first helical polypeptide consisting essentially of the sequence of IQN17 (SEQ ID NO: 1), which encompasses at least one mutation; providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline; providing a test composition; measuring, by capillary zone electrophoresis, the degree of complex formation between the first helical polypeptide and the second helical polypeptide in the presence of the test composition; and comparing the measured degree of complex formation to that between the first helical polypeptide encompassing a wild-type sequence, and the second helical polypeptide in the presence of the same test composition; wherein a reduction in the degree of complex formation between the first helical polypeptide encompassing at least one mutation and the second helical polypeptide in the presence of the test composition compared to the degree of complex formation between the first helical polypeptide encompassing a wild-type sequence and the second helical polypeptide in the presence of the same test composition identifies the susceptibility of the mutant gp41 fusion protein to a test composition.

Another embodiment of the invention is a kit for identifying a fusion inhibitor comprising: a first helical polypeptide consisting essentially of the sequence of IQN17 (SEQ ID NO: 1); a second helical polypeptide of 34 or less than 34 amino acids comprising the amino acid sequence W-X1-X2-W-X3-X4-X5-1, wherein X1, X2, X3, X4, and X5 are each independently chosen from any amino acid except proline.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is an example of a Capillary Electrophoresis system. A capillary zone electrophoresis apparatus comprises a high-voltage supply (A), electrodes (anode and cathode, B and C respectively), buffer (D), and a capillary tube (E). F corresponds to a Light source detector (280 nm), G corresponds to a photo-receptor, and H to a computer with recorder.

FIG. 2. Capillary Electrophoresis Electropherograms of IQN17 and C28. It shows the peaks of IQN17 and Alexa-C28 (C28*) run under same experimental conditions.

FIG. 3. Titration of 5 μM of Alexa-C28 with IQN17. The curves are off-set by both axis's for illustration purposes. At increasing concentration of IQN17, the amount of unbound IQN17, represented by the broader peak on the right, decreased from 100% to 0%.

FIG. 4. Inhibition of IQN17/C28 complex formation by C34 peptide. The concentration of IQN17 and Alexa-C28 was 3 and 10 μM respectively. At increasing concentrations of C34, greater amount of C28 became unbound because of the competitive binding of C34 to IQN17. This demonstrates the base of detection of potential fusion inhibitors that act like C34. The data at different concentrations of C34 are offset by both axis's for illustration purposes.

FIG. 5. Inhibition of IQN17/Alexa-C28 binding by C34 and C34 peptide mutants (W1A, M2A, W4A, 18A). All experiments were performed in CZE under same conditions and concentrations of peptides. The concentration of IQN17 and Alexa-C28 was 3 and 10 μM respectively.

### DETAILED DESCRIPTION OF THE INVENTION

These terms as used herein are defined as follows:

Helical polypeptide as used herein refers to a polypeptide with a helical content of at least 70% in aqueous solution, such as for example 74%, 80%, 85%, 90% and 95%. The percent helical content is estimated as previously described (Sreerama et al., Anal. Biochem. 209:32-44 (1993)).

A fusion inhibitor, as used herein, is any compound that prevents membrane fusion between target cells and free virus or viral infected cells. For example, a HIV fusion inhibitor may be any compound that binds to gp41 and prevents the fusogenic six-helical bundle formation, thus decreases gp41-mediated membrane fusion. In one embodiment, a fusion inhibitor is any compound that decreases the degree of complex formation or binding affinity. In another embodiment, a fusion inhibitor is chosen from peptides, derivatized peptides, C-peptides, D-peptides, N-peptides, cyclic or linear, small and large molecules that decrease gp41-mediated membrane fusion, including, for example, disrupting the complex formation of the N- and C-helices of gp41.

C-peptides are peptide segments derived from the second heptad repeat region of HIV gp41 sequence and their derivatives, including C34, C28, T20, and T1249.

N-peptides are peptide segments derived from the first heptad repeat region of HIV gp41 sequence and their derivatives, including N36 and DP107.

A test composition comprises any compound, including, but not limited to, peptides, dipeptides, tripeptides, polypeptides, proteins, small and large organic molecules and derivatives thereof. Large organic molecules are those with a molecular weight higher than 1000 Daltons.

Complex formation or binding affinity, as used herein, refers to the ability of at least two entities, for example, at least two peptides, to interact with one another, such as, for example, by hydrogen bonding and Van der Waals interactions. The degree of complex formation of two peptides would therefore be the extent of interaction between two peptides. This parameter ranges between 0-100%, with 100% being one peptide completely bound to the other peptide at the experimental concentrations.

The binding affinity of the first helical polypeptide and the second helical polypeptide, both alone and in the presence of the test composition, may be measured by any method known in the art. For example, the binding affinity may be measured by titrating the second helical polypeptide against a fixed concentration of the first helical polypeptide or vice versa.

The degree of complex formation measures the percentage of bound second helical polypeptide relative to the total amount of second helical polypeptide, at fixed concentrations of the first and second helical polypeptides. Although one can calculate binding affinity from the degree of complex formation, this is usually not recommended because of possible large errors. The difference between degree of complex formation and binding affinity is that binding affinity is usually determined by a series of measurements of degree of complex formation at a fixed concentration of one binding component, and at increasing concentrations of the second binding component until the first binding component is completely bound. A titration curve is thus obtained.

A label facilitates the separation and/or identification of a composition or compound including, for example, proteins. Examples of labels include, but are not limited to, radioactive labels; chromophores; fluorophores, fluorescent labels, such as rhodamine, Cy-3, Cy-5, tetramethyl rhodamine, lucifer yellow, C6-NBD, DIO-Cn-(3), BODIPY-FL, eosin, propidium iodide, Dil-Cn-(3), lissamine rhodamine B, Dil-Cn-(5), allophycocyanin, Texas red; ELISA type labels such as biotin; and enzymatic substrate type labels. Preferred fluorescent labels are those which do not induce nonspecific binding.

As described above, the invention comprises providing a first helical polypeptide comprising the sequence of IQN17 (SEQ ID NO: 1). The size of the first helical polypeptide may comprise any number of amino acids as long as the first helical polypeptide is able to bind to the second helical polypeptide described below. In one embodiment, the first helical polypeptide comprises less than 29 amino acids, such as for example less than 21 amino acids, and such as for example, less than 18 amino acids.

By the use of the phrase “consisting essentially of” in describing the sequences and compositions of the invention, it is meant to include any changes, variations, derivatives, additions, insertions, and mutations to the sequence of IQN17 and composition, that do not prohibit binding of the first helical polypeptide and the second helical polypeptide.

In one embodiment, examples of changes, variations, derivatives, and mutations to the sequence of IQN17 and composition include, for instance, sequences SEQ ID NO. 4 (W571A): RMKQIEDKIEEIESKQKKIENEIARIKKLLQLTVAGIKQLQARIL; SEQ ID NO. 5 (K574A): RMKQIEDKIEEIESKQKKIENEIARIKKLLQLTVWGIAQLQARIL; SEQ ID NO. 6 (Q577A): RMKQIEDKIEEIESKQKKIENEIARIKKLLQLTVWGIKQLAARIL: SEQ ID NO. 7 (R579A): RMKQIEDKIEEIESKQKKIENEIARIKKLLQLTVWGIKQLQAAIL.

In one embodiment, examples of insertions, and additions to the sequence of IQN17 and composition, include larger sequences that can be made by fusing the same GCN4-pI_{Q}I peptide to longer segments of gp41 sequences, in particular to the N36-17 residue segment and more residues toward the N-terminal of gp41, such as --XLLQLTVWGIKQLQARIL, or one can also add more residues toward the C-terminal of gp41 of the 17 residue segment, such as --LLQLTVWGIKQLQARILX. The symbol “--” illustrates the place of union with the GCN4-pI_{Q}I peptide. For these examples of insertions and additions, X refers to the sequence of aminoacids from gp41 flanking the two ends of the N36 17-residue segment. Said sequence of aminoacids comprises one or more aminoacids. Said sequences further include any changes, variations, derivatives, additions, insertions, and mutations as long as they not prohibit binding of the first helical polypeptide and the second helical polypeptide.

In one embodiment, the invention comprises providing a first helical polypeptide consisting essentially of a sequence having a sequence identity of at least about 90% with said IQN17, such as for example 90%, 91%, 92%, 93%, 94%, 95%, 96%, 97%, 98%, 99%, 100%.

Sequence identity, or homology, is defined as a sequence modified with substitutions, insertions, deletions, gaps and the like known to those skilled in the art so that the function or activity of the sequence is not destroyed. For example, in reference to the first helical polypeptide, a homologous polypeptide of the helical polypeptides still permits detectable binding of the first helical polypeptide and the second helical polypeptide. Sequence identity may be determined using algorithms known to the person skilled in the art such as FASTA and BLAST. Alternatively, the degree of homology or sequence identity between two sequences may be evaluated by a direct comparison of amino acid sequences.

Other examples of the first helical polypeptide include, but are not limited to mutations and variations of IQN17.

The first helical polypeptide may, for example, be provided at a concentration ranging from about 0 μM to about 1 mM, or for example, at a concentration ranging from about 1 μM to about 4 μM.

The invention also comprises providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline, which disrupts helix formation. In one embodiment, the second helical polypeptide of less than 34 amino acids consists essentially of the amino acid sequence W-X1-X2-W-X3-X4-X5-I, wherein X1, X2, X3, X4, and X5 are each independently chosen from any amino acid except proline. In one embodiment, the amino acid sequence W-X1-X2-W-X3-X4-X5-I is WMEWDREI (SEQ ID NO: 2). Each one of X1, X2, X3, X4, X5 is an amino acid different from proline, and any such amino acid is not restricted as to being different from or the same as any other such amino acid. In other words, Xᵢ and Xⱼ, i≠j, are selected in such a manner that Xᵢ=Xⱼ or Xᵢ≠Xⱼ, as long as Xᵢ and Xⱼ are not proline, for i, j=1, 2, 3, 4, 5. This selection is concisely referred herein as “wherein X1, X2, X3, X4, and X5 are each independently chosen from any amino acid except proline”.

The second helical polypeptide may also comprise the amino acid sequence of C28. The second helical polypeptide may alternatively consist essentially of the amino acid sequence of C28, WMEWDREINNYTSLIHSLIEESQNQQEK (SEQ ID NO: 3). The second helical polypeptide may as well consist of the amino acid sequence of C28, WMEWDREINNYTSLIHSLIEESQNQQEK (SEQ ID NO: 3).

The second helical polypeptide may also, for example, comprise the amino acid sequence W-X1-X2-W-X3-X4-X5-I, wherein the W and I residues are at the a and d positions of the helix of this second helical polypeptide. See, e.g., Cole and Garsky, Biochemistry, 40, 5633-5641 (2001).

As described above, the X1, X2, X3, X4, and X5 of the amino acid sequence, W-X1-X2-W-X3-X4-X5-I, may each, independently, be chosen from any amino acid except proline. For example, in one embodiment, X1, X2, X3, X4, and X5 may be chosen from Ala, Asx, Cys, Asp, Glu, Phe, Gly, His, IlE, Lys, Leu, Met, Asn, Gln, Arg, Ser, Thr, Val, Trp, Tyr, and Glx. Optionally, X1, X2, X3, X4, and X5, in another embodiment, may be chosen from non-natural amino acids as long as they do not affect helix formation, such as trans-dimethyl 1-aminocyclopentane-1,3-dicarboxylic acid, dimethyl L-glutamic acid, ethyl cycloleucine, N-tosyl-1-aminocyclopentane-trans-1,3-dicarboxylic acid.

The second helical polypeptide may, for example, be provided at a concentration ranging from about 0 μM to about 1 mM, or for example at a concentration ranging from about 1 μM to about 11 μM.

In one embodiment, the first helical polypeptide consists essentially of the amino acid sequence of SEQ ID NO: 1 and the second helical polypeptide consists essentially of the amino acid sequence of SEQ ID NO: 2. In another embodiment, the first helical polypeptide consists essentially of the amino acid sequence of SEQ ID NO: 1 and the second helical polypeptide consists essentially of the amino acid sequence of SEQ ID NO: 3.

In another embodiment, the first helical polypeptide consists of the amino acid sequence of SEQ ID NO: 1 and the second helical polypeptide consists of the amino acid sequence of SEQ ID NO: 2. In another embodiment, the first helical polypeptide consists of the amino acid sequence of SEQ ID NO: 1 and the second helical polypeptide consists of the amino acid sequence of SEQ ID NO: 3.

In the practice of the methods of the invention, a test composition is also provided. In one embodiment, the test composition may comprise a peptide, such as C-peptides, D-peptides, N-peptides, linear or cyclic. In another embodiment, the test composition may comprise, for example, dipeptides, tripeptides, polypeptides, and derivatives thereof. In another embodiment the test composition may comprise small and large organic molecules.

The skilled artisan will appreciate that the assay can be performed with one or more first helical polypeptides, with one or more second helical polypeptides, with one or more test compositions.

In one embodiment, the degree of complex formation of the first helical polypeptide and the second helical polypeptide is measured. The experiment may then be repeated in the presence of the test composition and the degree of complex formation of the experiments compared. Of course, the order of the testing may be varied or the degree of complex formation of the first helical polypeptide and the second helical polypeptide may be known and used as a base to compare to the degree of complex formation in the presence of a test composition. The methods of the invention may also be used as or comprise part of a high-throughput screening assay where numerous test compositions are evaluated for their affect on the degree of complex formation of the first helical polypeptide and the second helical polypeptide. In addition, the methods of the invention may be employed to evaluate resistance of the envelope gene of different viral mutants. Thus, the first helical polypeptide, such as IQN17, N36, and N-peptides, may encompass mutations, and its ability to bind to the second helical polypeptide be evaluated as described above.

In one embodiment of the invention, the first helical polypeptide, the second helical polypeptide and the test composition may be labeled.

Methods of measuring degree of complex formation or binding affinity include any method that has sufficient sensitivity to detect changes in the degree of binding of the first helical polypeptide and the second helical polypeptide. In one embodiment, the preferred method of measuring binding is capillary zone electrophoresis.

Capillary zone electrophoresis may, for example, provide greater sensitivity and detection limits, and requires small amount of samples. The method can be used further to measure the inhibition of binding affinity or complex formation of the first helical polypeptide and the second helical polypeptide in the presence of a test composition. The half inhibition concentration (IC₅₀) can range between 1 nM and 500 μM. For example, a high sensitivity method such as capillary zone electrophoresis may allow detection of both the bound and unbound forms of at least one of the first helical polypeptide and second helical polypeptide. In another embodiment, a high sensitive method such as capillary zone electrophoresis may detect small changes in binding affinity and/or weak binders, e.g. molecules with a small dissociation constant, K_{D}.

Other conventional methods for indirect in vitro detection of affinity between fusion inhibitors and gp41 proteins, include, but are not limited to, regular electrophoresis, isothermal titration calorimetry, fluoresence resonance energy transfer (FRET), phage display, chromatographic affinity methods, circular dichroism, direct fluoresence assays, ELISA type assays, NMR deuterium-proton exchange, enzymatic or chemical residue modification methods. In another embodiment, the binding affinity may be measured by isothermal titration calorimetry.

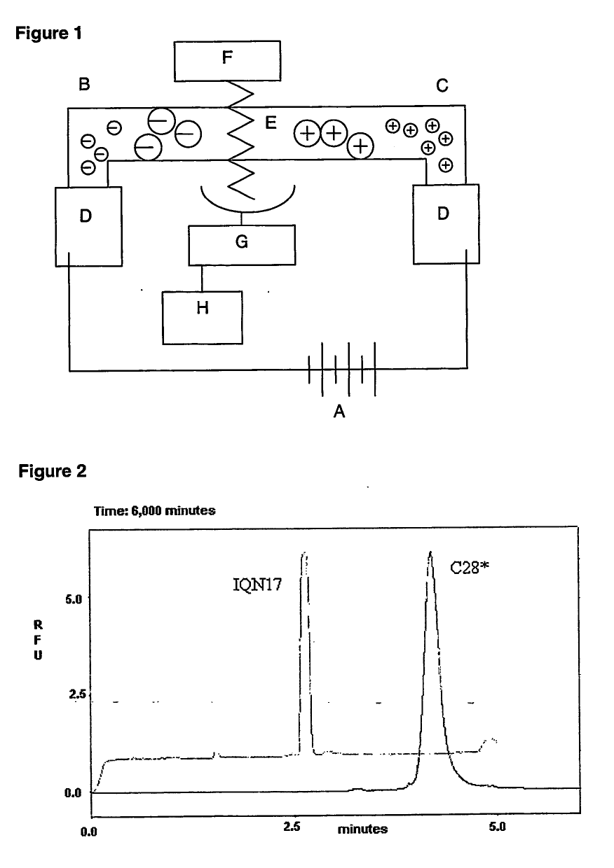

In a preferred embodiment, the binding affinity is measured by capillary zone electrophoresis as shown, for example, in FIG. 1. Electrophoresis is a separation technique that is based on the mobility of ions in an electric field. Positively charged ions migrate towards a negative electrode and negatively-charged ions migrate toward a positive electrode. Performing electrophoresis in small-diameter capillaries allows the use of very high electric fields because the small capillaries efficiently dissipate the heat that is produced. Increasing the electric fields produces very efficient separations and reduces separation times. A capillary zone electrophoresis apparatus comprises a high-voltage supply (A in FIG. 1), electrodes (anode and cathode, B and C in FIG. 1), buffer (D), and a capillary tube (E). Detection in capillary zone electrophoresis include amongst others, absorbance, fluorescence, electrochemical, and mass spectrometry. In FIG. 1, F corresponds to a Light source detector (280 nm), G corresponds to a photo-receptor, and H to a computer with recorder. The size of the capillary for use in capillary zone electrophoresis may also dictate other parameters. For example, in one embodiment, a capillary of inner diameter of 75 μm may be used, and variations on the size of the capillary may change other conditions of the assay. The length of the capillaries may vary between 5 and 100 cm, preferably between 10 cm and 80 cm, more preferably between 20 and 55 cm. The capillaries may be made of fused silica. Preferably the silica is derivatized to include a hydrophobic coating material. Capillaries precoated with polyacrylamide or polyvinylalcohol may also be employed.

In one embodiment, the pH range used in the methods of the invention may need to be adjusted to provide the necessary conditions for binding of the first helical polypeptide and the second helical polypeptide. In one embodiment, the pH ranges from about 5 to about 9, such as for example, about 8.5.

The invention also encompasses fusion inhibitors identified by the methods of the invention and reports that are generated comprising a listing, analysis, or other information regarding a fusion inhibitor identified by the methods of the invention. Another embodiment of the invention is a kit comprising a first helical polypeptide consisting essentially of the sequence of IQN17 (SEQ ID NO: 1); and a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline. The kit may further comprise any reagents necessary to practice the methods of the invention and any equipment or apparatus needed to practice the methods of the invention, such as the equipment necessary to measure binding affinity or degree of complex formation.

As one of skill in the art would immediately recognize, further tests that could be used to confirm whether a test compound is an effective gp41-mediated membrane fusion inhibitor may also be performed, including but not limited to cellular assays.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits, ordinary rounding approaches, and known and understood errors and variations in measurements being reported and claimed.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The order of the steps of the methods of the invention may, of course, be varied. One of skill in the art would be able to determine which variations in the order of the steps is applicable.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

1. Competitive Binding Assay Using Capillary Zone Electrophoresis:

The binding affinity of a 28-residue peptide from the second heptad region of GP41 (C28) was measured using a chimeric peptide (IQN17) that contains a segment of GCN4 at the N-terminal and 17 residues of the first heptad repeat region of HIV-1 GP-41 at the C-terminal. Eckert et al., Cell 99, 103-115 (1999). C28 was labeled with the fluorescent molecule, Alexa-430 (Molecular Probes) at its carboxyl terminal (Alexa-C28). The binding was measured by titration of labeled C28 with IQN17. The concentration of bound and unbound C28 was measured by capillary zone electrophoresis.

Reagents and buffers. Sodium Borate and Boric Acid were purchased from Sigma. Binding and separation buffers were identical. Buffers were prepared by mixing equal weights of sodium borate and boric acid in ultrapure water (Ω<16 MΩ) and adjusting pH to 8.5. Dimethyl sulfoxide was purchased from Sigma.

Peptides. IQN-17 has been described elsewhere. Eckert et al., Cell 99, 103-115 (1999). IQN-17 was purchased from Anaspec, Inc. Thirteen peptides were synthesized on a Rainin 430A peptide synthesizer using Fmoc/TBTU chemistry and dimethylformamide as solvent. After cleavage from the resin, peptides were purified by reverse phase high performance liquid chromatography (Varian, Inc.) on a C18 Vydac preparative column using water-acetonitrile gradient in the presence of 0.05% trifluoracetic acid and then lyophilized. The expected molecular weights of all peptides were verified by MALDI-TOF on a Voyager-DE BioSpectrometry Work Station and then analyzed on analytical HPLC (Varian, Inc.) for sample homogeneity. The molecular weights of peptides were confirmed. Peptide C28 was fluorescently labeled using Alexa-430 from Molecular Probes.

Capillary Electrophoresis Separations. Capillary electrophoresis experiments were conducted on a Beckman Coulter P/ACE System MDQ and a Spectrumedix 9610HTS. The capillaries used in the Beckman Coulter had an inner diameter of 75 μm, 50 cm of effective length, and inner surface of fused silica. Separations were conducted with an applied voltage of 30 kV. The capillaries used in the Spectrumedix 9610HTS has an inner diameter of 50 μm, effective length of 50 cm, and an inner surface of fused silica. Separations were conducted with an applied voltage of 13 kV. Separation buffer was 20 mM Sodium Borate, pH=8.5. Peptides traveling passed a fluorescence detector were excited by a laser emitting at 488 nm. The peaks were analyzed using software packages supplied by the instrument companies. The data for individual runs of IQN17 and Alexa-C28 are shown in FIG. 2. In the figures, RFU refers to relative fluorescence units.

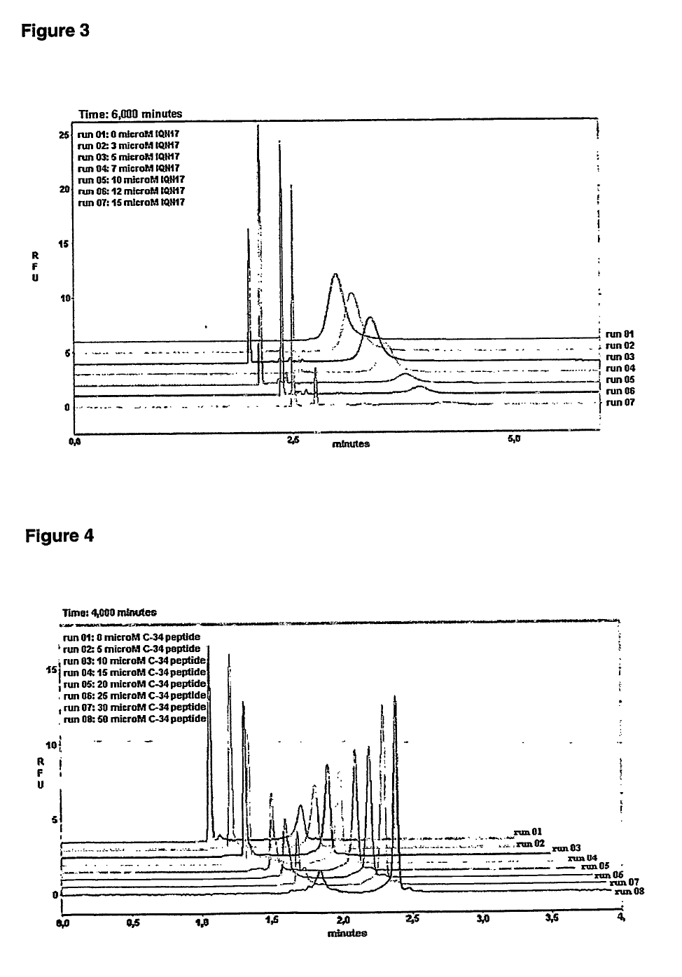

Binding of C28 to IQN17: Peptide concentrations were determined by weight. Peptides were dissolved in binding buffer. The binding affinity of C28 to IQN-17 was determined by a capillary electrophoresis (FIG. 3). Alexa-C28 and IQN-17 peptides were allowed to bind for at least one hour prior to measurement by CZE.

The area of the Alexa-C28 peaks in increasing concentrations of IQN-17 was analyzed in comparison to the area of Alexa-C28 in the absence of IQN-17 by capillary zone electrophoresis. At 3 μM C28 and 8 μm IQN17, about 80% C28 was bound to IQN17.

Inhibiting Binding of C28 by C34 and C34 mutants: Three component binding assays measured the ability of C34 based peptides (C34, W1A, M2A, W4A, 18A) to displace Alexa-C28 from a binding site on IQN-17.

| | |
|---|---|
| C34 | WMEWDREINNYTSLIHSLIEESQNQQEKNEQELL |
| W1A | AMEWDREINNYTSLIHSLIEESQNQQEKNEQELL |
| M2A | WAEWDREINNYTSLIHSLIEESQNQQEKNEQELL |
| W4A | WMEADREINNYTSLIHSLIEESQNQQEKNEQELL |
| I8A | WMEWDREANNYTSLIHSLIEESQNQQEKNEQELL |

Peptides were dissolved in binding buffer except for C-34 and C-34 mutants which were dissolved in dimethyl sulfoxide. Binding was measured in solutions of 3 μM Alexa-C28 and 8 μM IQN-17 unless otherwise noted. Buffer, IQN-17, a C-34-based peptide, and Alexa-C28 were mixed in that order. DMSO was added to bring the concentration in the final solution to 5% by volume. The three peptides were allowed to bind for at least one hour prior to measurement by CZE. The areas of the Alexa-C28 peaks at a constant concentration of IQN-17 and varying concentrations of C-34-based peptide was analyzed in comparison to the area of Alexa-C28 in the absence of C-34 and IQN-17.

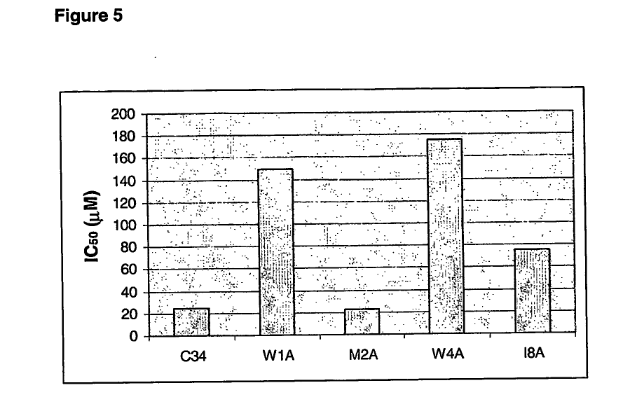

For unlabeled peptides, the amount that inhibits binding of 50% of Alexa-C28 to IQN17 gives the IC50 value for that peptide (FIG. 4 and FIG. 5). Experiments performed in the context of this invention showed that binding to the hydrophobic pocket is preferably accomplished when the terminal of C34 contains two tryptophans. The same experiments indicated that the presence of isoleucin at the 8^{th} position of C34 contributes less to such binding compared with the binding when two tryptophans are present at the N-terminal of C34. Furthermore, it was observed in experiments performed in the context of this invention that the inhibition capacity is in some embodiments not affected by the mutation of the second methionine to alanine. One explanation consistent with this observation is that this residue plays little or no role in binding to IQN17.

1. A method of identifying a fusion inhibitor comprising:
providing a first helical polypeptide consisting essentially of the sequence of IQN17 (SEQ ID NO: 1);
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring the degree of complex formation between the first helical polypeptide and the second helical polypeptide in the presence of the test composition using capillary zone electrophoresis; and
comparing the measured degree of complex formation to the degree of complex formation between the first helical polypeptide and the second helical polypeptide in the absence of the test composition to determine if the test composition is a fusion inhibitor.

2. A method of identifying a fusion inhibitor comprising:
providing a first helical polypeptide comprising a sequence of IQN17 (SEQ ID NO: 1);
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring the degree of complex formation between the first helical polypeptide and the second helical polypeptide in the presence of the test composition using capillary zone electrophoresis; and
comparing the measured degree of complex formation to the degree of complex formation between the first helical polypeptide and the second helical polypeptide in the absence of the test composition to determine if the test composition is a fusion inhibitor.

3. A method of identifying inhibitors of gp41-mediated membrane fusion comprising:
providing a first helical polypeptide consisting essentially of the sequence of IQN17 (SEQ ID NO: 1);
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring the degree of complex formation between the first helical polypeptide and the second helical polypeptide in the presence of the test composition using capillary zone electrophoresis at a pH ranging from about 5 to about 9; and
comparing the measured degree of complex formation to the degree of complex formation between the first helical polypeptide and the second helical polypeptide in the absence of the test composition;
wherein a reduction in the degree of complex formation between the first helical polypeptide and the second helical polypeptide in the presence of the test composition compared to the degree of complex formation between the first helical polypeptide and the second helical polypeptide in absence of the test composition identifies the test composition as an inhibitor of gp41-mediated membrane fusion.

4. A method of identifying inhibitors of gp41-mediated membrane fusion comprising:
providing a first helical polypeptide comprising a sequence of IQN17 (SEQ ID NO: 1);
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring the degree of complex formation between the first helical polypeptide and the second helical polypeptide in the presence of the test composition using capillary zone electrophoresis at a pH ranging from about 5 to about 9; and
comparing the measured degree of complex formation to the degree of complex formation between the first helical polypeptide and the second helical polypeptide in the absence of the test composition;
wherein a reduction in the degree of complex formation between the first helical polypeptide and the second helical polypeptide in the presence of the test composition compared to the degree of complex formation between the first helical polypeptide and the second helical polypeptide in absence of the test composition identifies the test composition as an inhibitor of gp41-mediated membrane fusion.

5. A method of identifying the mechanism of inhibition of a fusion inhibitor comprising:
providing a first helical polypeptide consisting essentially of the sequence of IQN17 (SEQ ID NO: 1);
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring, by capillary electrophoresis, the degree of complex formation between the first helical polypeptide and the second helical polypeptide in the presence of the test composition; and
comparing the measured degree of complex formation to the degree of complex formation between the first helical polypeptide and the second helical polypeptide in the presence of a different test composition to determine if the first test composition has the same or different mechanism of inhibition.

6. A method for measuring resistance of mutant gp41 fusion proteins to a test composition comprising:
providing a first helical polypeptide consisting essentially of the sequence of IQN17 (SEQ ID NO: 1), which encompasses at least one mutation;
providing a second helical polypeptide of 34 or less than 34 amino acids comprising the amino acid sequence W-X1-X2-W-X3-X4-X5-I, wherein X1, X2, X3, X4, and X5 are each independently chosen from any amino acid except proline;
providing a test composition;
measuring, by capillary zone electrophoresis, the degree of complex formation between the first helical polypeptide and the second helical polypeptide in the presence of the test composition; and
comparing the measured degree of complex formation to the degree of complex formation between the first helical polypeptide encompassing a wild-type sequence, and the second helical polypeptide in the presence of the same test composition;
wherein a reduction in the degree of complex formation between the first helical polypeptide, encompassing at least one mutation, and the second helical polypeptide in the presence of the test composition compared to the degree of complex formation between the first helical polypeptide encompassing a wild-type sequence and the second helical polypeptide in the presence of the same test composition identifies the susceptibility of the mutant gp41 fusion protein to a test composition.

7. The method of claim 1, wherein measuring off the degree of complex formation between the first helical polypeptide and the second helical polypeptide in the presence of the test compositions is performed by using capillary zone electrophoresis with laser-induced fluorescence detection.

8. The method of claim 1, wherein the first helical polypeptide comprises less than 29 amino acids.

9. The method of claim 1, wherein the first helical polypeptide comprises less than 21 amino acids.

10. The method of claim 1 to 6, wherein the first helical polypeptide comprises less than 18 amino acids.

11. The method of claim 1 to 6, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

12. The method of claim 1 to 6, wherein the second helical polypeptide comprises the amino acid sequence of SEQ ID NO: 2.

13. The method of claim 1 to 6, wherein the first helical polypeptide further comprises a label.

14. The method of claim 1, wherein the second helical polypeptide further comprises a label.

15. The method of claim 13, wherein the label is a fluorescent label.

16. The method of claim 1, wherein binding affinity is measured instead of the degree of complex formation, by titrating a first helical polypeptide against a fixed concentration of a second helical polypeptide.

17. The method of claim 1, wherein the test composition comprises a peptide.

18. The methods of claim 17, wherein the peptide is any one chosen from C-peptides, D-peptides, and N-peptides.

19. The methods of claims 18, wherein the peptides are linear or cyclic.

20. The method of claim 1, wherein the test composition comprises a small molecule.

21. The method of claim 1, wherein the test composition comprises a large molecule.

22. A report comprising a fusion inhibitor identified by the method of claim 1.

23. A report comprising the mechanism of action of a fusion inhibitor identified by the method of claim 5.

24. A report comprising the resistance of the least one fusion inhibitor identified by the method of claim 6.

25. A fusion inhibitor identified by the method of claim 1.

26. The method of claim 2, wherein measuring off the degree of complex formation between the first helical polypeptide and the second helical polypeptide in the presence of the test compositions is performed by using capillary zone electrophoresis with laser-induced fluorescence detection.

27. The method of claim 3, wherein measuring off the degree of complex formation between the first helical polypeptide and the second helical polypeptide in the presence of the test compositions is performed by using capillary zone electrophoresis with laser-induced fluorescence detection.

28. The method of claim 4, wherein measuring off the degree of complex formation between the first helical polypeptide and the second helical polypeptide in the presence of the test compositions is performed by using capillary zone electrophoresis with laser-induced fluorescence detection.

29. The method of claim 5, wherein measuring off the degree of complex formation between the first helical polypeptide and the second helical polypeptide in the presence of the test compositions is performed by using capillary zone electrophoresis with laser-induced fluorescence detection.

30. The method of claim 6, wherein measuring off the degree of complex formation between the first helical polypeptide and the second helical polypeptide in the presence of the test compositions is performed by using capillary zone electrophoresis with laser-induced fluorescence detection.

31. The method of claim 2, wherein the first helical polypeptide comprises less than 29 amino acids.

32. The method of claim 3, wherein the first helical polypeptide comprises less than 29 amino acids.

33. The method of claim 4, wherein the first helical polypeptide comprises less than 29 amino acids.

34. The method of claim 5, wherein the first helical polypeptide comprises less than 29 amino acids.

35. The method of claim 6, wherein the first helical polypeptide comprises less than 29 amino acids.

36. The method of claim 2, wherein the first helical polypeptide comprises less than 21 amino acids.

37. The method of claim 3, wherein the first helical polypeptide comprises less than 21 amino acids.

38. The method of claim 4, wherein the first helical polypeptide comprises less than 21 amino acids.

39. The method of claim 5, wherein the first helical polypeptide comprises less than 21 amino acids.

40. The method of claim 6, wherein the first helical polypeptide comprises less than 21 amino acids.

41. The method of claim 2, wherein the first helical polypeptide comprises less than 18 amino acids.

42. The method of claim 3, wherein the first helical polypeptide comprises less than 18 amino acids.

43. The method of claim 4, wherein the first helical polypeptide comprises less than 18 amino acids.

44. The method of claim 5, wherein the first helical polypeptide comprises less than 18 amino acids.

45. The method of claim 6, wherein the first helical polypeptide comprises less than 18 amino acids.

46. The method of claim 2, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

47. The method of claim 3, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

48. The method of claim 4, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

49. The method of claim 5, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

50. The method of claim 6, wherein the second helical polypeptide comprises the amino acid sequence W-X1-X2-W-X3-X4-X5-I, wherein X1, X2, X3, C4, X5 are amino acids selected from Ala, Asx, Cys, Asp, Glu, Phe, Gly, His Iie, Lys, Leu, Met, Asn, Gln, Arg, Ser, Thr, Val, Trp, Tyr, Glx, and non-natural amino acids.

51. The method of claim 2, wherein the second helical polypeptide comprises the amino acid sequence of SEQ ID NO: 2.

52. The method of claim 3, wherein the second helical polypeptide comprises the amino acid sequence of SEQ ID NO: 2.

53. The method of claim 4, wherein the second helical polypeptide comprises the amino acid sequence of SEQ ID NO: 2.

54. The method of claim 5, wherein the second helical polypeptide comprises the amino acid sequence of SEQ ID NO: 2.

55. The method of claim 6, wherein the second helical polypeptide comprises the amino acid sequence of SEQ ID NO: 2.

56. The method of claim 2, wherein the first helical polypeptide further comprises a label.

57. The method of claim 3, wherein the first helical polypeptide further comprises a label.

58. The method of claim 4, wherein the first helical polypeptide further comprises a label.

59. The method of claim 5, wherein the first helical polypeptide further comprises a label.

60. The method of claim 6, wherein the first helical polypeptide further comprises a label.

61. The method of claim 2, wherein the second helical polypeptide further comprises a label.

62. The method of claim 3, wherein the second helical polypeptide further comprises a label.

63. The method of claim 4, wherein the second helical polypeptide further comprises a label.

64. The method of claim 5, wherein the second helical polypeptide further comprises a label.

65. The method of claim 6, wherein the second helical polypeptide further comprises a label.

66. The method of claim 14, wherein the label is a fluorescent label.

67. The method of claim 2, wherein binding affinity is measured instead of the degree of complex formation, by titrating a first helical polypeptide against a fixed concentration of a second helical polypeptide.

68. The methos of claim 3, wherein binding affinity is measured instead of the degree of complex formation, by titrating a first helical polypeptide against a fixed concentration of a second helical polypeptide.

69. The method of claim 4, wherein binding affinity is measured instead of the degree of complex formation, by titrating a first helical polypeptide against a fixed concentration of a second helical polypeptide.

70. The method of claim 5, wherein binding affinity is measured instead of the degree of complex formation, by titrating a first helical polypeptide against a fixed concentration of a second helical polypeptide.

71. The method of claim 6, wherein binding affinity is measured instead of the degree of complex formation, by titrating a first helical polypeptide against a fixed concentration of a second helical polypeptide.

72. The method of claim 2, wherein the test composition comprises a peptide.

73. The method of claim 3, wherein the test composition comprises a peptide.

74. The method of claim 4, wherein the test composition comprises a peptide.

75. The method of claim 5, wherein the test composition comprises a peptide.

76. The method of claim 6, wherein the test composition comprises a peptide.

77. The method of claim 2, wherein the test composition comprises a small molecule.

78. The method of claim 3, wherein the test composition comprises a small molecule.

79. The method of claim 4, wherein the test composition comprises a small molecule.

80. The method of claim 5, wherein the test composition comprises a small molecule.

81. The method of claim 6, wherein the test composition comprises a small molecule.

82. The method of claim 2, wherein the test composition comprises a large molecule.

83. The method of claim 3, wherein the test composition comprises a large molecule

84. The method of claim 4, wherein the test composition comprises a large molecule

85. The method of claim 5, wherein the test composition comprises a large molecule

86. The method of claim 6, wherein the test composition comprises a large molecule

87. A report comprising a fusion inhibitor identified by the method of claim 2.

88. A report comprising a fusion inhibitor identified by the method of claim 3.

89. A report comprising a fusion inhibitor identified by the method of claim 4.


SEQUENCE LISTING (14 sequences)

SEQ ID NO: 1 - protein, 45 aa, Artificial Sequence
  Mixture of GCN4-PIQI and N36 gp41 terminal of HIV
RMKQIEDKIEEIESKQKKIENEIARIKKLLQLTVWGIKQLQARIL

SEQ ID NO: 2 - protein, 8 aa, Artificial Sequence
  Synthetic Construct
WMEWDREI

SEQ ID NO: 3 - protein, 28 aa, Artificial Sequence
  Synthetic Construct
WMEWDREINNYTSLIHSLIEESQNQQEK

SEQ ID NO: 4 - protein, 45 aa, Artificial Sequence
  Synthetic Construct
RMKQIEDKIEEIESKQKKIENEIARIKKLLQLTVAGIKQLQARIL

SEQ ID NO: 5 - protein, 45 aa, Artificial Sequence
  Synthetic Construct
RMKQIEDKIEEIESKQKKIENEIARIKKLLQLTVWGIAQLQARIL

SEQ ID NO: 6 - protein, 45 aa, Artificial Sequence
  Synthetic Construct
RMKQIEDKIEEIESKQKKIENEIARIKKLLQLTVWGIKQLAARIL

SEQ ID NO: 7 - protein, 45 aa, Artificial Sequence
  Synthetic Construct
RMKQIEDKIEEIESKQKKIENEIARIKKLLQLTVWGIKQLQAAIL

SEQ ID NO: 8 - protein, 18 aa, Artificial Sequence
  Synthetic Construct
XLLQLTVWGIKQLQARIL

SEQ ID NO: 9 - protein, 18 aa, Artificial Sequence
  Synthetic Construct
LLQLTVWGIKQLQARILX

SEQ ID NO: 10 - protein, 34 aa, Artificial Sequence
  Synthetic Construct
WMEWDREINNYTSLIHSLIEESQNQQEKNEQELL

SEQ ID NO: 11 - protein, 34 aa, Artificial Sequence
  Synthetic Construct
AMEWDREINNYTSLIHSLIEESQNQQEKNEQELL

SEQ ID NO: 12 - protein, 34 aa, Artificial Sequence
  Synthetic Construct
WAEWDREINNYTSLIHSLIEESQNQQEKNEQELL

SEQ ID NO: 13 - protein, 34 aa, Artificial Sequence
  Synthetic Construct
WMEADREINNYTSLIHSLIEESQNQQEKNEQELL

SEQ ID NO: 14 - protein, 34 aa, Artificial Sequence
  Synthetic Construct
WMEWDREANNYTSLIHSLIEESQNQQEKNEQELL